(19) United States
(12) Patent Application Publication
(10) Pub. No.: US 2008/0086089 A1
(43) Pub. Date: Apr. 10, 2008

(54) MAGNETICALLY INDUCED SAFETY TECHNOLOGY

(76) Inventors: S. Ray Isaacson, Roy, UT (US); Mark A. Crawford, Sandy, UT (US)

Correspondence Address: David W. Highet;Becton, Dickinson and Company, Mail Code 089, 1 Becton Drive, Franklin Lakes, NJ, 07417-1880, US

(21) Appl. No.: 11/456,248

(22) Filed: Jul. 10, 2006

(51) Int. Cl. A61M 5/178

(52) U.S. Cl. 604/164.08

(57) ABSTRACT

Safety medical devices that secure needle tips in the devices using magnetic forces are provided. Magnetic force is used to rotate binding components of safety devices. Safety needle assemblies and methods of making and using the same are provided. Safety needle assemblies are provided, comprising: a cannula comprising a proximal end and a distal end; a hub disposed on the proximal end of the cannula; a housing partially surrounding a portion of the cannula, the housing comprising a distal end; and a magnetic latch that is operative to secure the distal end of the cannula within the housing. A magnetic latch comprises a first magnetic material that is located on or within the binding component and a second magnetic material that can interact with the first material. As the needle tip is retracted into the safety housing and the tip clears an end sense system of the binding component, the attracting or repelling force of the magnetic latch causes the binding component to rotate and secure the needle tip within the housing.

### FIELD

This application relates to safety medical devices, in particular, to devices that secure needle tips within the devices.

### BACKGROUND

Dangers associated with accidental needle sticks are well-known. Safe handling of used needles is important to minimize transmission of harmful contaminants or infectious diseases. Many safety products today use binding as a method of securing a needle tip in a safety device to reduce the occurrence of accidental needle sticks. One way to secure a needle tip is to use a binding component that bears down on the cannula when pressure is applied off-center from the axis of the needle. This pressure forces an aperture in the binding component to bite down on the cannula, preventing the binding component from translating. When this occurs, the needle can be held in one position, generally within the confines of a housing, and accidental needle sticks can be avoided.

In some examples, the binding component contains a feature that stores mechanical energy (e.g., the coil springs of U.S. Pat. Nos. 6,719,737 and 6,695,819 and the leaf spring of U.S. Patent Publication No. 2005/0182362) that bears down on the cannula and locks it in place. Other examples use frictional-based binding. Some components, however, have limited shelf lives due to, for example, relaxation of the springs. Other components are cumbersome and require relatively large housing components. In some instances, the stored mechanical devices are relatively slow in acting to bind the needle tips.

There is a need, therefore, for binding components that are reduced in size, are faster acting, and have longer shelf-lives.

### SUMMARY

In one aspect of the present invention, safety needle assemblies are provided that comprise a cannula comprising a proximal end and a distal end; a hub disposed on the proximal end of the cannula; a housing partially surrounding a portion of the cannula, the housing comprising a distal end; and a magnetic latch that is operative against the cannula to secure the distal end of the cannula within the housing.

In another aspect of the present invention, a method of making a safety needle assembly is provided, the method comprising: providing a cannula comprising a proximal end and a distal end; disposing a hub on the proximal end of the cannula; partially surrounding a portion of the cannula with a housing comprising a distal end; and locating a magnetic latch such that it is operative against the cannula.

In a further aspect of the present invention, a method of securing a needle tip is provided, the method comprising: moving a safety device from a proximal end of a cannula towards the needle tip located at a distal end, wherein the safety device comprises a housing and a magnetic latch; moving the end sense member past the needle tip toward the distal end of the cannula; and rotating the binding component with magnetic force, thereby securing the needle tip within the housing.

### BRIEF DESCRIPTION OF THE DRAWINGS

FIGS. 1 and 2 show needle assemblies according to one aspect of the invention.

FIG. 3 shows a cross-section view of a housing of a needle assembly according to one aspect of the invention.

FIGS. 4-8 show a schematic view of the interior area of a housing according to an aspect of the invention.

FIGS. 9-11 show a schematic view of the interior area of a housing according to an aspect of the invention.

### DETAILED DESCRIPTION

Safety medical devices, in particular, devices that secure needle tips in the devices using magnetic forces are provided. In some embodiments, the use of magnetic forces to bind needles can be faster-acting and more reliable than the use of stored mechanical energy. In other embodiments, safety devices using magnetic forces, which are not subject to components that experience mechanical relaxation, generally have longer shelf-lives than devices using stored mechanical energy. Moreover, in some embodiments, the use of magnetic latches permits safety device housings to be smaller, for example, shorter in axial length and smaller in diameter size, than devices using coils, for example. Reference to magnetic latches includes systems that use magnetically activated components, that is, components of magnetic latches move due to magnetic forces.

Reference to a magnetic material is meant to include a material that produces a magnetic field external to itself. Examples of magnetic materials include, but are not limited to ferrites, namely, iron oxides; rare earth metals; and other metals, for example, iron, nickel, and cobalt.

Generally, with friction-based binding technology, where friction arms are used to provide resistance needed to cause a binding plate to rotate, the plate is not allowed to rotate until an end-sensing arm of the plate reaches the end of a cannula. When the arm reaches the end of the cannula, the plate is rotated because it is being pushed to the distal end of the cannula only on one side of the binding plate. The friction arms are there to ensure that the plate rotates and “bites” into the cannula, locking the safety to the needle. Without the frictional arms, a safety device could slide off the end of the cannula if the plate does not rotate properly.

With the magnetic energy-based technology of the present invention, magnets are used to provide positive rotation in a binding component. The magnetic energy is used to push or pull the binding component off-center to ensure that it rotates and bites or binds a cannula. Typically, a magnetic energy safety device has no frictional arms present in the safety housing for storing mechanical energy, although it may be desirable to use a magnetic energy safety device in conjunction with friction-based binding. As an example, when the tip of a binding component having a front smooth tip reaches the end of the cannula, magnetic energy between, for example, the binding component and an area within a safety housing, causes the latch to rotate and bite on the cannula. The housing then presses against the binding component ensuring that it bites and locks on the cannula, thereby refraining the safety device from coming off of the needle tip. By reference to cannula, it is understood that cannulas of unlimited gauge sizes and needle tip geometries can be used. Also, safety devices of the present invention can be attached to many types of needle assemblies. Examples include, without limitation, short needles, long needles, needles attached to intravenous catheters, epidural needles, spinal needles, needles having luer adapters, syringes, and the like. In addition, a cannula having more than one diameter can be used in conjunction with embodiments of the present invention.

In some devices in accordance with the magnetic-energy technology of the present invention, the devices generally resist being retracted, re-exposing the needle tip. This is based on the forces of, for example, the magnet and housing combination pushing on the binding component on the opposite side during an attempted safety retraction. The binding component bites down into the cannula generally in the same orientation as when trying to slide it off the distal end. In examples where a transverse barrier is part of the binding component, the transverse barrier captures the needle tip and presents itself over the tip, not allowing the safety device to be retracted.

In one aspect of the present invention, safety needle assemblies are provided that comprise a cannula comprising a proximal end and a distal end; a hub disposed on the proximal end of the cannula; a housing partially surrounding a portion of the cannula, the housing comprising a distal end; and a magnetic latch that is operative against the cannula to secure the distal end of the cannula within the housing.

In some embodiments, the housing may have an open distal end. In this case, in some examples, a cover that partially surrounds a portion of the cannula can be associated with the housing in order to form an interior area. A cover, as desired, serves to close off the distal end of the housing. One example of a cover is a cap which is engagable with the housing. Another example of the cover is a portion of a can or sleeve that surrounds the exterior of the housing and closes off the distal end of the housing. The can or sleeve can be physically separate from the housing.

In an embodiment, the magnetic latch comprises a first magnetic material that is located in sufficient proximity to a second magnetic material to repel or attract the second magnetic material; and a binding component partially surrounding the cannula, the binding component comprising the second magnetic material.

In one embodiment, the assembly is free of a friction arm. By reference to free of a friction arm it is meant that a component used to store mechanical energy that has a close fit against the cannula is not present. Having a safety device that is free of friction arms eases assembly, for example, by reducing the number of parts that need to be handled or that are subject to damage during assembly. In addition, tactile response by users can also be improved.

The binding component can be made of any material, for example, metals or plastics. If the metal or plastic used does not have magnetic properties, then the binding component can be coated with magnetic material. In other cases, it may be desirable to use a separately fabricated magnetic material that is attached to the binding component.

In one embodiment, the second magnetic material is dispersed in a coating that is adhered to the binding component. In another embodiment, the second magnetic material is physically separate from the binding component and is attached to the binding component.

In some examples, the binding component further comprises an end sense member. In other examples, the binding component further comprises a transverse barrier. The second magnetic material can be located within or on the end sense member.

In one embodiment, the first magnetic material is attached to the cover. In another embodiment, the first magnetic material is dispersed in a coating that is adhered to a portion of the interior area. In yet another embodiment, the first magnetic material is attached to an interior surface of the housing. By interior surface of the housing, it is meant the surface of the housing that faces the cannula. In another embodiment, the first magnetic material is located on the exterior of the housing.

In certain examples, the first magnetic material and the second magnetic material attract each other. In other examples, the first magnetic material and the second magnetic material repel each other.

In one embodiment, the first magnetic material is attached to an interior surface of the housing, the binding component comprises an end sense member, and the first magnetic material attracts the second magnetic material. In another embodiment, the first magnetic material is attached to an interior surface of the housing, the binding component comprises an end sense member and a transverse barrier, and the first magnetic material attracts the second magnetic material.

In a further embodiment, the first magnetic material is attached to the cover; the binding component further comprises a transverse barrier; the second magnetic material is attached to the binding component; and the first magnetic material repels the second magnetic material.

In an embodiment, the first magnetic material is attached to the cover; the binding component comprises an end sense member; the second magnetic material is located within or on the end sense member; and the first magnetic material attracts the second magnetic material.

In one embodiment, the first magnetic material is attached to an interior surface of the housing; the binding component comprises an end sense member; the second magnetic material is located within or on the end sense member; and the first magnetic material repels the second magnetic material.

In a further embodiment, the first magnetic material comprises an electromagnet and a battery. In another embodiment, the safety needle assembly using an electromagnet and battery, further comprises a safety mechanism operatively associated with the housing having a first position that permits the electromagnet to be de-energized and a second position that permits the electromagnet to be energized. In one example, the safety mechanism comprises a switch.

In another aspect of the present invention, a method of making a safety needle assembly is provided, the method comprising: providing a cannula comprising a proximal end and a distal end; disposing a hub on the proximal end of the cannula; partially surrounding a portion of the cannula with a housing comprising a distal end; and locating a magnetic latch such that it is operative against the cannula.

In a further aspect of the present invention, a method of securing a needle tip is provided, the method comprising: moving a safety device from a proximal end of a cannula towards the needle tip located at a distal end, wherein the safety device comprises a housing and a magnetic latch; moving the end sense member past the needle tip toward the distal end of the cannula; and rotating the binding component with magnetic force, thereby securing the needle tip within the housing. In one example, the movement of the binding component along the cannula is substantially friction-free. By substantially friction-free it is meant that an aperture of the binding component is loose against the cannula. In this regard, an embodiment of the present invention can be substantially friction-free while using an end sense member because the aperture of the binding component can remain loose against the cannula. In terms of end sensing, it may be desirable to use features, for example, ferrule marks, bumps, notches, welded catches, and the like, on the cannula.

Before describing several exemplary embodiments of the invention, it is to be understood that the invention is not limited to the details of construction or process steps set forth in the following description. The invention is capable of other embodiments and of being practiced or being carried out in various ways.

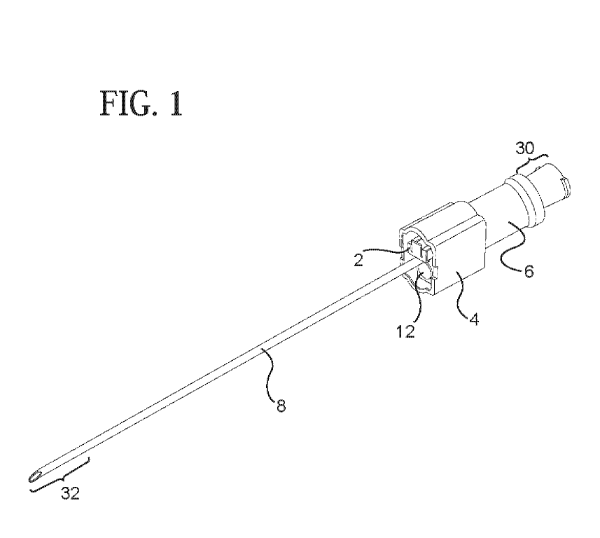

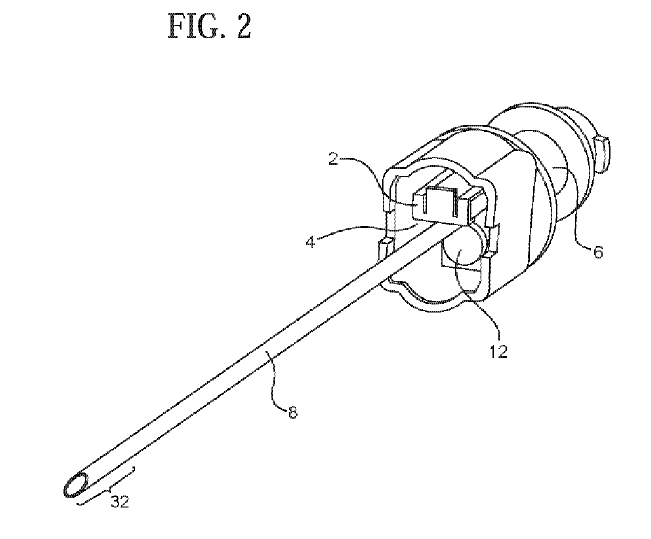

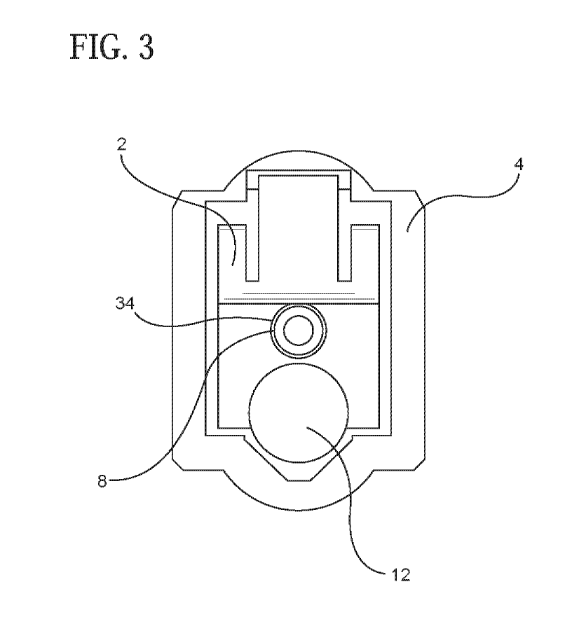

Turning to the figures, wherein like reference numerals refer to like elements, FIGS. 1 and 2 show needle assemblies and FIG. 3 shows a cross-section view across a housing of a needle assembly. In one example, a magnetic latch comprises a binding component 2, also referred to as a binding plate, having magnetic properties and a magnet 12. The magnet 12, in this example, is located within the safety housing 4. In other examples, it may be desirable to locate a magnet outside of the safety housing. A hub 6 attaches to a proximal end 30 of a cannula 8 and partially surrounds the safety housing 4 and the cannula 8. In operation, the cannula 8 slides through an aperature 34.

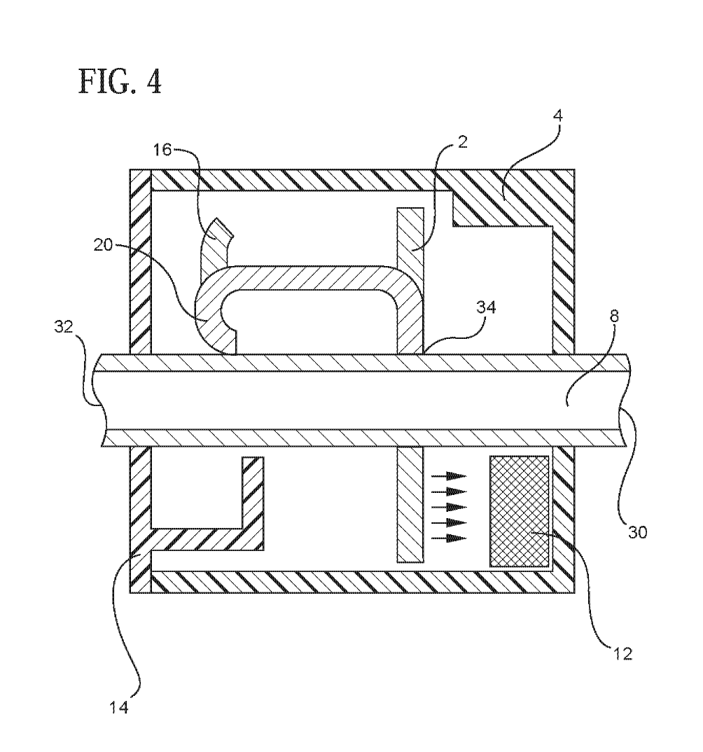

FIG. 4 shows a schematic of the interior of the safety housing of an embodiment where the binding component 2 has an aperture 34, a transverse barrier 16, and an end sense member 20. In this example, the binding component 2 is made of material that is magnetically reactive. The safety cap 14 engages with the safety housing 4. A magnet 12 is located on the safety housing 4. The magnetically reactive binding component 2 is attracted to magnet 12. In practice, when the safety device is in position on the proximal end 30 of the cannula 8, the binding component 2 is substantially perpendicular to the longitudinal axis of the cannula 8, and therefore the edges of aperture 34 do not bite the cannula 8, due to the force of the end sense member 20 on the cannula 8. This is true even though the binding component 2 is attracted to magnet 12. When the safety device is moved from a first position at the proximal end 30 towards a second position at the distal end 32, the cannula 8 slides through the aperture 34. Once the needle tip is retracted into the safety device and the end sense member 20 clears the tip, the binding component 2 is no longer held in place by the end sense member 20 and the attracting magnetic force causes the binding component 2 to rotate and the edges of aperture 34 to bite down on the cannula 8. In this position, the transverse barrier 16 covers the tip.

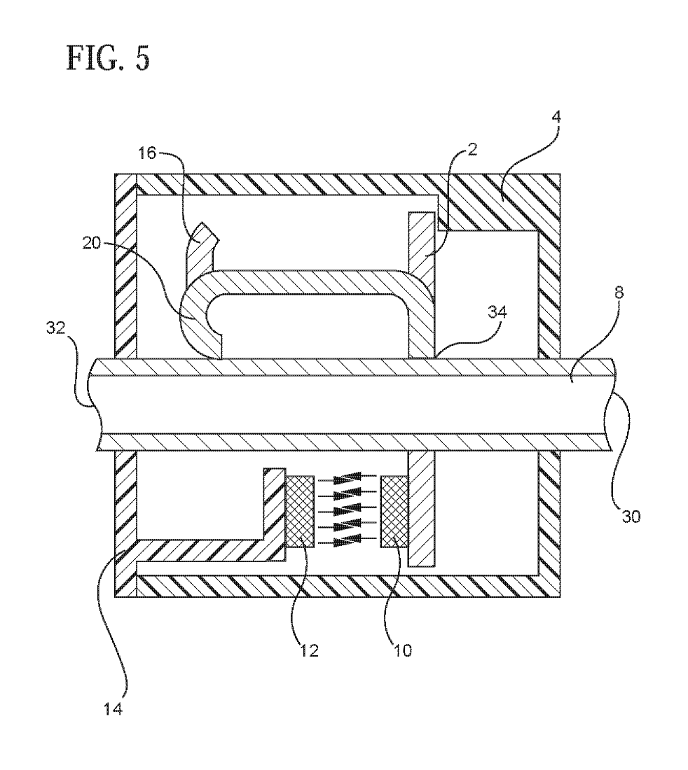

FIG. 5 shows a schematic of the interior of the safety housing of an embodiment where a magnet 10 is attached to the binding component 2 having an aperture 34, a transverse barrier 16, and an end sense member 20. The safety cap 14 engages with the safety housing 4. In this example, the binding component 2 is made of material that is substantially magnetically un-reactive. A magnet 12 is attached to the safety cap 14. In this example, magnet 10 is repelled by magnet 12. In practice, when the safety device is in position on the proximal end 30 of the cannula 8, the binding component 2 is substantially perpendicular to the longitudinal axis of the cannula 8, and therefore the edges of aperture 34 do not bite the cannula 8, due to the force of the end sense member 20 on the cannula 8. This is true even though the magnet 10 located on the binding component 2 is repelled by magnet 12. When the safety device is moved from a first position at the proximal end 30 towards a second position at the distal end 32, the cannula 8 slides through the aperture 34. Once the needle tip is retracted into the safety device and the end sense member 20 clears the tip, the binding component 2 is no longer held in place by the end sense member 20 and the repelling magnetic force causes the binding component 2 to rotate and the edges of aperture 34 to bite down on the cannula 8. In this position, the transverse barrier 16 covers the tip.

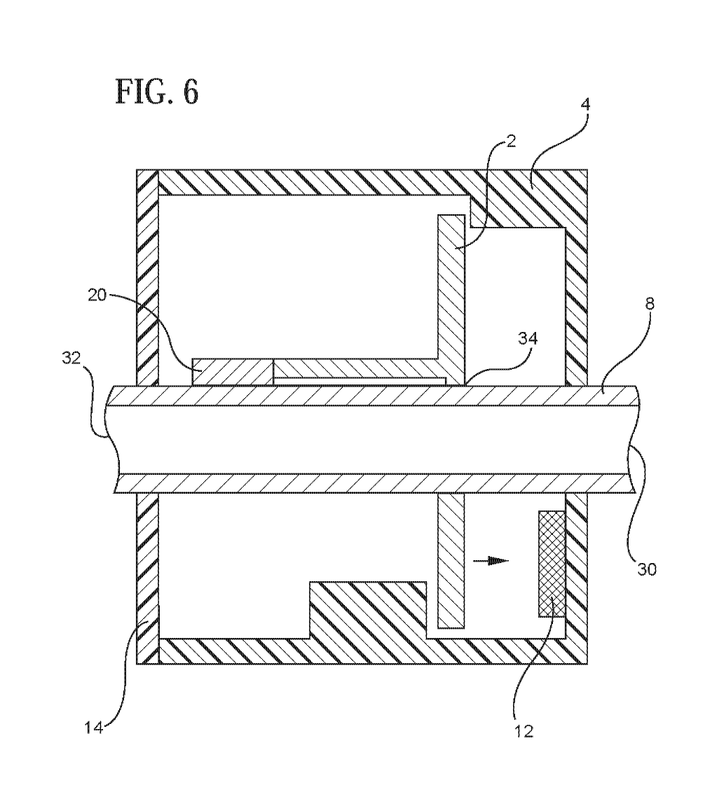

FIG. 6 shows a schematic of the interior of the safety housing of an embodiment where the binding component 2 has an end sense member 20. The safety cap 14 engages with the safety housing 4. In this example, the binding component 2 is made of material that is magnetically reactive. A magnet 12 is located on the safety housing 4. In this example, the binding component 2 is attracted to magnet 12. In practice, when the safety device is in position on the proximal end 30 of the cannula 8, the binding component 2 is substantially perpendicular to the longitudinal axis of the cannula 8, and therefore the edges of aperture 34 do not bite the cannula 8, due to the force of the end sense member 20 on the cannula 8. This is true even though the binding component 2 is attracted to magnet 12. When the safety device is moved from a first position at the proximal end 30 towards a second position at the distal end 32, the cannula 8 slides through the aperture 34. Once the needle tip is retracted into the safety device and the end sense member 20 clears the tip, the binding component 2 is no longer held in place by the end sense member 20 and the attracting magnetic force causes the binding component 2 to rotate and the edges of aperture 34 to bite down on the cannula 8.

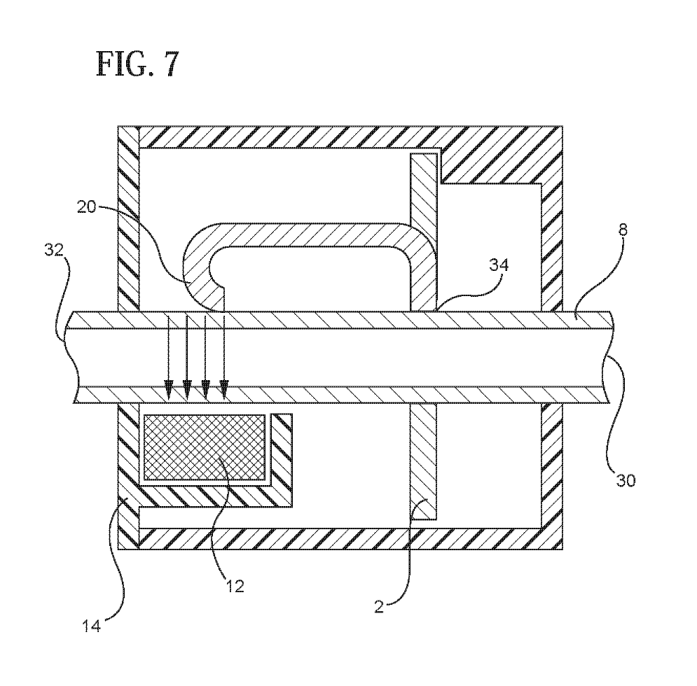

FIG. 7 shows a schematic of the interior of the safety housing of an embodiment where the binding component 2 has an end sense member 20. In this example, the end sense member portion of the binding component is made of material that is magnetically reactive. The safety cap 14 engages with the safety housing 4. A magnet 12 is located on the safety cap 14. The magnetically reactive end sense member 20 is attracted to magnet 12. In practice, when the safety device is in position on the proximal end 30 of the cannula 8, the binding component 2 is substantially perpendicular to the longitudinal axis of the cannula 8, and therefore the edges of aperture 34 do not bite the cannula 8, due to the force of the end sense member 20 on the cannula 8. This is true even though the end sense member 20 of the binding component 2 is attracted to magnet 12. When the safety device is moved from a first position at the proximal end 30 towards a second position at the distal end 32, the cannula 8 slides through the aperture 34. Once the needle tip is retracted into the safety device and the end sense member 20 clears the tip, the binding component 2 is no longer held in place by the end sense member 20 and the attracting magnetic force causes the binding component 2 to rotate and the edges of aperture 34 to bite down on the cannula 8.

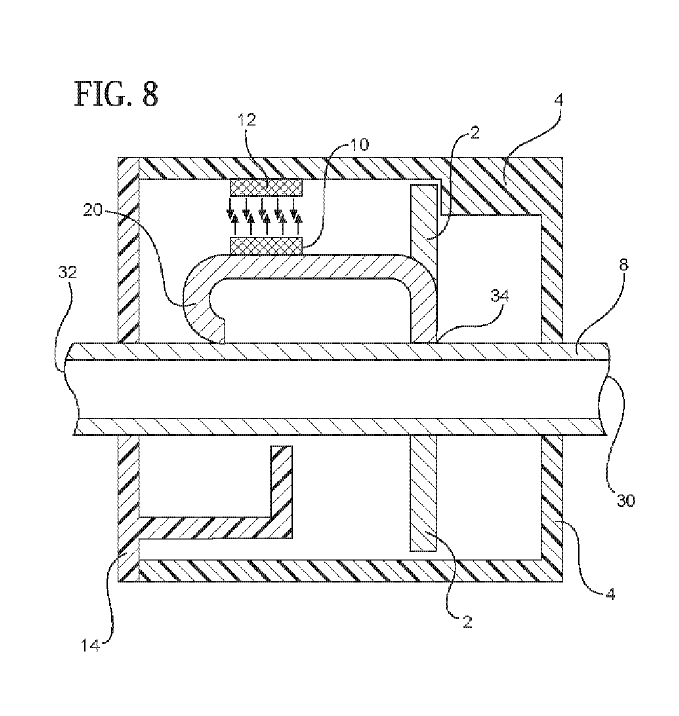

FIG. 8 shows a schematic of the interior of the safety housing of an embodiment where a magnet 10 is attached to the end sense member 20 of the binding component 2. The safety cap 14 engages with the safety housing 4. In this example, the binding component 2 is made of material that is substantially magnetically un-reactive. A magnet 12 is attached to the safety housing 4. In this example, magnet 10 is repelled by magnet 12. In practice, when the safety device is in position on the proximal end 30 of the cannula 8, the binding component 2 is substantially perpendicular to the longitudinal axis of the cannula 8, and therefore the edges of aperture 34 do not bite the cannula 8, due to the force of the end sense member 20 on the cannula 8. This is true even though the magnet 10 on the binding component 2 is repelled by magnet 12. When the safety device is moved from a first position at the proximal end 30 towards a second position at the distal end 32, the cannula 8 slides through the aperture 34. Once the needle tip is retracted into the safety device and the end sense member 20 clears the tip, the binding component 2 is no longer held in place by the end sense member 20 and the repelling magnetic force causes the binding component 2 to rotate and the edges of aperture 34 to bite down on the cannula 8.

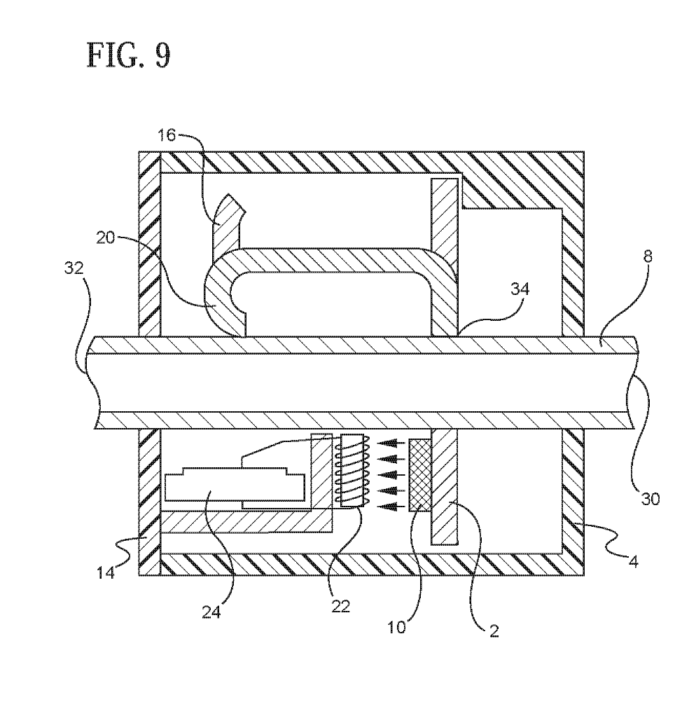

FIG. 9 shows a schematic of the interior of the safety housing of an embodiment where a magnet 10 is attached to the binding component 2 having a transverse barrier 16 and an end sense member 20. The safety cap 14 engages with the safety housing 4. In this example, the binding component 2 is made of material that is substantially magnetically un-reactive. An electromagnet 22 is located on the safety cap 14. A battery 24 is connected to the electromagnet 22. In this example, magnet 10 is repelled by electromagnet 22. In practice, when the safety device is in position on the proximal end 30 of the cannula 8, the binding component 2 is substantially perpendicular to the longitudinal axis of the cannula 8, and therefore the edges of aperture 34 do not bite the cannula 8, due to the force of the end sense member 20 on the cannula 8. This is true even though the magnet 10 on the binding component 2 is repelled by magnet 12. When the safety device is moved from a first position at the proximal end 30 towards a second position at the distal end 32, the cannula 8 slides through the aperture 34. Once the needle tip is retracted into the safety device and the end sense member 20 clears the tip, the binding component 2 is no longer held in place by the end sense member 20 and the repelling magnetic force causes the binding component 2 to rotate and the edges of aperture 34 to bite down on the cannula 8. In this position, the transverse barrier 16 covers the tip.

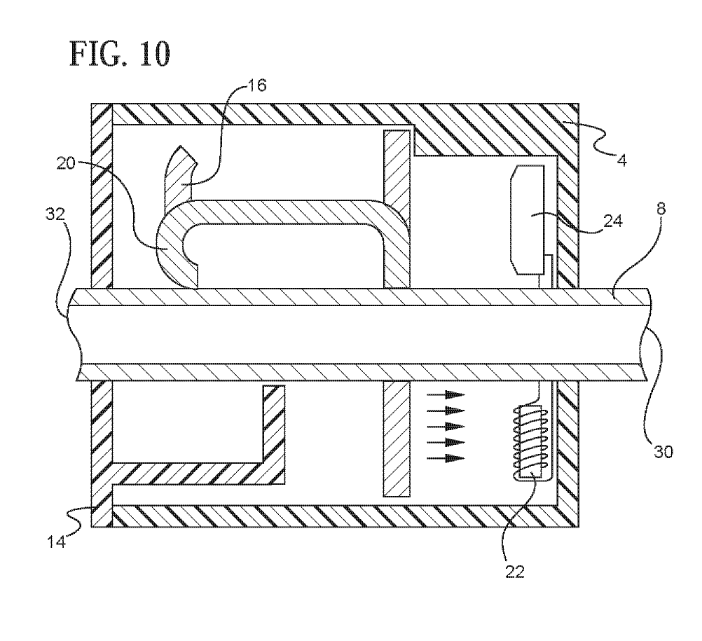

FIG. 10 shows a schematic of the interior of the safety housing of an embodiment where the binding component 2 has a transverse barrier 16 and an end sense member 20. The safety cap 14 engages with the safety housing 4. In this example, the binding component 2 is made of material that is magnetically reactive. An electromagnet 22 is located on the safety housing 4. A battery 24 is connected to the electromagnet 22. In this example, the binding component 2 is attracted to the electromagnet 22. In practice, when the safety device is in position on the proximal end 30 of the cannula 8, the binding component 2 is substantially perpendicular to the longitudinal axis of the cannula 8, and therefore the edges of aperture 34 do not bite the cannula 8, due to the force of the end sense member 20 on the cannula 8. This is true even though the binding component 2 is attracted to electromagnet 22. When the safety device is moved from a first position at the proximal end 30 towards a second position at the distal end 32, the cannula 8 slides through the aperture 34. Once the needle tip is retracted into the safety device and the end sense member 20 clears the tip, the binding component is no longer held in place by the end sense member 20 and the attracting magnetic force causes the binding component 2 to rotate and the edges of aperture 34 to bite down on the cannula 8. In this position, the transverse barrier 16 covers the tip.

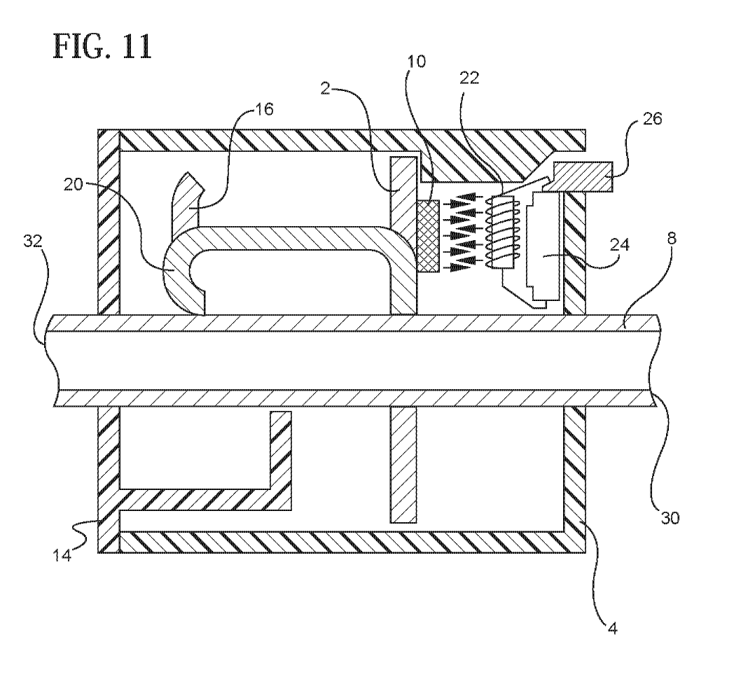

FIG. 11 shows a schematic of the interior of the safety housing of an embodiment where the binding component 2 has a transverse barrier 16 and an end sense member 20. A magnet 10 is attached to the binding component 2. The safety cap 14 engages with the safety housing 4. In this example, the binding component 2 is made of material that is substantially magnetically un-reactive. An electromagnet 22 is attached to a battery 24 which is attached to the safety housing 4.

A safety mechanism comprising a switch 26 is provided in this example. In a first position, for example, when the safety device is located at the proximal end 30 of the cannula and the safety housing 4 is engaged with the hub (not shown), the switch 26 interrupts the electrical circuit between the battery 24 and the electromagnet 22. In a second position, the switch 26 is no longer in contact with the battery circuit, permitting the electrical circuit to be completed and causing the electromagnet to energize. The switch 26 can be in any form that is amenable to disrupting the electrical circuit between the battery and electromagnet. For example, the switch may be a piece integral to the needle assembly hub. In another example, it may be desirable that the switch is separate from the needle assembly hub. One example of this is a disposable piece of plastic that can be slid away from the safety housing in order to active the electromagnet. Prior to engaging the safety mechanism, the needle assembly of this embodiment is generally friction-free. In this example, magnet 10 is repelled by electromagnet 22.

In practice, when the safety device is in position on the proximal end 30 of the cannula 8, the binding component 2 is substantially perpendicular to the longitudinal axis of the cannula 8, and therefore the edges of aperture 34 do not bite the cannula 8, due to the force of the end sense member 20 on the cannula 8. At this point, there is no interaction between the magnet 10 on the binding component 2 and the electromagnet 22 because the electrical circuit is interrupted by switch 26. When the safety device is moved from a first position at the proximal end 30 towards a second position at the distal end 32, the cannula 8 slides through the aperture 34. Once the needle tip is retracted into the safety device and the end sense member 20 clears the tip, the binding component is no longer held in place by the end sense member. The electrical circuit engages when switch 26 is no longer interrupting the circuit. If the switch is an integral part of the hub, for example, then the movement of the safety housing 4 towards the distal end 32 causes the switch 26 to disengage from the circuit. If the switch is a separate piece of plastic, for example, then the circuit is completed upon removal of the plastic by, for example, a practitioner. Then, the repelling magnetic force causes the binding component 2 to rotate and the edges of aperture 34 to bite down on the cannula 8. In this position, the transverse barrier 16 covers the tip.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed:

1. A safety needle assembly comprising:
a cannula comprising a proximal end and a distal end;
a hub disposed on the proximal end of the cannula;
a housing partially surrounding a portion of the cannula, the housing comprising a distal end; and
a magnetic latch that is operative against the cannula to secure the distal end of the cannula within the housing.

2. The safety needle assembly of claim 1, wherein the magnetic latch comprises a first magnetic material that is located in sufficient proximity to a second magnetic material to repel or attract the second magnetic material; and a binding component partially surrounding the cannula, the binding component comprising the second magnetic material.

3. The safety needle assembly of claim 2, wherein the first magnetic material is attached to an interior surface of the housing.

4. The safety needle assembly of claim 2, wherein the first magnetic material is attached to an exterior surface of the housing.

5. The safety needle assembly of claim 2, further comprising a cover located at the distal end of the housing and partially surrounds a portion of the cannula, wherein the first magnetic material is attached to the cover.

6. The safety needle assembly of claim 2, wherein the first magnetic material is dispersed in a coating that is adhered to a portion of an interior surface of the housing.

7. The safety needle assembly of claim 2, wherein the first magnetic material is dispersed in a coating that is adhered to a portion of an exterior surface of the housing.

8. The safety needle assembly of claim 2, wherein the second magnetic material is dispersed in a coating that is adhered to a portion of the binding component.

9. The safety needle assembly of claim 2, wherein the second magnetic material is physically separate from the binding component and is attached to the binding component.

10. The safety needle assembly of claim 9, further comprising a cover located at the distal end of the housing and partially surrounds a portion of the cannula, wherein the first magnetic material is attached to the cover.

11. The safety needle assembly of claim 9, wherein the first magnetic material is attached to an interior surface of the housing.

12. The safety needle assembly of claim 9, wherein the first magnetic material is attached to an exterior surface of the housing.

13. The safety needle assembly of claim 2, wherein the first magnetic material and the second magnetic material attract each other.

14. The safety needle assembly of claim 2, wherein the first magnetic material and the second magnetic material repel each other.

15. The safety needle assembly of claim 2, wherein the binding component further comprises an end sense member.

16. The safety needle assembly of claim 15, wherein the second magnetic material is located within or on the end sense member.

17. The safety needle assembly of claim 2, wherein the binding component further comprises a transverse barrier.

18. The safety needle assembly of claim 2 that is free of a friction arm.

19. The safety needle assembly of claim 3, wherein the binding component further comprises an end sense member; and the first magnetic material attracts the second magnetic material.

20. The safety needle assembly of claim 19, wherein the binding component further comprises a transverse barrier.

21. The safety needle assembly of claim 5, wherein the binding component further comprises a transverse barrier; the second magnetic material is attached to the binding component; and the first magnetic material repels the second magnetic material.

22. The safety needle assembly of claim 5, wherein the binding component further comprises an end sense member; the second magnetic material is located within or on the end sense member; and the first magnetic material attracts the second magnetic material.

23. The safety needle assembly of claim 3, wherein the binding component further comprises an end sense member; the second magnetic material is located within or on the end sense member; and the first magnetic material repels the second magnetic material.

24. The safety needle assembly of claim 2, wherein the first magnetic material comprises an electromagnet and a battery.

25. The safety needle assembly of claim 19, wherein the first magnetic material comprises an electromagnet and a battery.

26. The safety needle assembly of claim 21, wherein the first magnetic material comprises an electromagnet and a battery.

27. The safety needle assembly of claim 24, further comprising a safety mechanism operatively associated with the housing having a first position that permits the electromagnet to be de-energized and a second position that permits the electromagnet to be energized.

28. The safety needle assembly of claim 27, wherein the first magnetic material is attached to an interior surface of the housing; the binding component further comprises a transverse barrier; the second magnetic material is attached to the binding component; and the first magnetic material repels the second magnetic material.

29. The safety needle assembly of claim 24 wherein the safety mechanism comprises a switch.

30. A method of making a safety needle assembly comprising:
providing a cannula comprising a proximal end and a distal end;
disposing a hub on the proximal end of the cannula;
partially surrounding a portion of the cannula with a housing comprising a distal end; and
locating a magnetic latch such that it is operative against the cannula.

31. A method of securing a needle tip comprising:
moving a safety device from a proximal end of a cannula towards the needle tip located at a distal end, wherein the safety device comprises a housing and a magnetic latch;
moving a portion of the magnetic latch past the needle tip toward the distal end of the cannula; and
rotating the binding component with magnetic force, thereby securing the needle tip within the housing.

32. The method of claim 31 wherein the portion of the magnetic latch that moves past the needle tip comprises an end sense member of a binding component.

33. The method of claim 31 wherein the movement of the binding component along the cannula is substantially friction-free.
